(19) United States
(12) Patent Application Publication
(10) Pub. No.: US 2002/0123709 A1
(43) Pub. Date: Sep. 5, 2002

(54) Orthotic device and method for making same

(76) Inventors: E. Marlowe Goble, Alta, WY (US); Alan Chervitz, Palm Harbor, FL (US); T. Wade Fallin, Hyde Park, UT (US); Robert W. Hoy, Logan, UT (US)

Correspondence Address: Pandiscio & Pandiscio, 470 Totten Pond Road, Waltham, MA, 02451-1914, US

(21) Appl. No.: 09/798,301

(22) Filed: Mar. 2, 2001

(51) Int. Cl. A61F 13/00

(52) U.S. Cl. 602/41

(57) ABSTRACT

An individual custom orthodic device for application to a limb of a mammal includes an inner sleeve for disposition on the limb, reinforcing strands fixed to the sleeve, and an outer sleeve fixed to and disposed around the inner sleeve. The inner and outer sleeve define therebetween a space in which a curable resin is disposed. upon curing of the resin, the device is hardened and is conformed to the limb.

### FIELD OF THE INVENTION

The invention relates to orthotic devices of the type applied to the limbs of mammals and is directed more particularly to an individualized custom device which closely conforms to the limb of a patient.

### BACKGROUND OF THE INVENTION

Injuries, particularly ankle injuries, to workers and athletes are common. Often the injuries are slight or moderate sprains of ankle ligaments. However slight, such injuries can be debilitating, rendering continued work afoot, or any athletic activity, quite painful.

In efforts to prevent such injuries and, when the injuries occur, to permit rapid return of an injured patient to the work place or athletic field, support devices have been devised, particularly ankle supports, which range from completely taping the ankle and foot to the application of rigid side support members, often with a rigid stirrup portion extending beneath the foot. The former is expensive, time consuming, and labor intensive. The latter usually is ill-fitting and uncomfortable, and often includes the use of a number of straps which must be manually connected together with a proper amount of tension.

There is a need for an orthotic support device which can be applied to an ankle or other affected body area without the use of straps, or the like, and which provides a close, comfortable fit.

Accordingly, an object of the invention is to provide an orthotic device which can be closely conformed to an ankle and foot, or other body area, and which provides required support to afford protection from injury or, if injury has already occurred, to afford comfortable support to the injured body area.

A further object of the invention is to provide such a device which can be applied to the patient by the patient, and which takes little time to apply, conform and fix to the body.

### SUMMARY OF THE INVENTION

With the above and other objects in view, as will hereinafter appear, a feature of the present invention is the provision of an orthotic device for application to a limb of a mammal, the device comprising a sleeve for disposition on the limb, reinforcing strands fixed to the sleeve, and a curable layer of resin disposed on the sleeve and the strands. Upon curing of the resin, the device is conformed to the limb and contains the reinforcing strands therein.

In accordance with a further feature of the invention, there is provided an orthotic device for application to a limb of a mammal, the device comprising an inner sleeve for disposition on the limb, reinforcing strands fixed to the sleeve, an outer sleeve fixed to and disposed around the inner sleeve, the inner and outer sleeves defining a space therebetween. A curable resin is disposed in the space, such that upon curing of the resin the device is conformed to the limb.

In accordance with a further feature of the invention, there is provided a method for forming a custom orthotic device, the method comprising the steps of providing a sleeve of elastomeric material, tacking reinforcing fiber strands to the sleeve, positioning the sleeve around a limb of a mammal, applying a layer of curable resin on the sleeve and over the reinforcing strands, and curing the resin, whereby the resin is made to harden conforming to the limb.

In accordance with a still further feature of the invention, there is provided a method for forming a custom orthotic device, the method comprising the steps of providing an inner sleeve of elastomeric material, tacking reinforcing fiber strands to the sleeve, fixing an outer sleeve of elastomeric material onto and around the inner sleeve such that the inner and outer sleeves define a space therebetween, positioning the device thus formed around a limb of a mammal, injecting curable liquid resin into the space, and curing the resin, whereby to harden the resin conforming to the limb.

The above and other features of the invention, including various novel details of construction and combinations of parts and method steps, will now be more particularly described with reference to the accompanying drawings and pointed out in the claims. It will be understood that the particular devices and methods embodying the invention are shown by way of illustration only and not as limitations of the invention. The principles and features of this invention may be employed in various and numerous embodiments without departing from the scope of the invention.

### BRIEF DESCRIPTION OF THE DRAWINGS

Reference is made to the accompanying drawings in which are shown illustrative embodiments of the invention, from which its novel features and advantages will be apparent.

In the drawings:

FIG. 1 is a plan view of one form of a panel for forming a sleeve portion of an orthotic device, with reinforcing strands fixed thereto;

FIG. 2 is a perspective view of the panel of FIG. 1 with edges thereof joined together to form a sleeve portion, with additional reinforcing strands fixed thereto;

FIG. 2A is similar to FIG. 2, but is an illustration of an alternative embodiment of the sleeve portion;

FIG. 3 is a perspective view of an orthotic device comprising the sleeve portion and reinforcing strands of FIG. 2 covered with a resinous material;

FIG. 4 is a perspective view of the orthotic device of FIG. 3 subjected to a heat or ultra violet source to cure the resin, to provide one form of the orthotic device illustrative of an embodiment of the invention;

FIG. 5 is a plan view of one form of an outer panel for forming an outer sleeve portion of an alternative orthotic device;

FIG. 6 is a perspective view of the outer panel of FIG. 5 with edges thereof joined together to form an outer sleeve portion which is fitted over the-device of FIG. 2;

FIG. 7 is a perspective view illustrating injection of a resin material into a gap defined by the inner and outer sleeves; and

FIG. 8 is a perspective view similar to FIG. 4 but showing the orthotic device of FIG. 7 subjected to the heat or ultra violet source to cure the resin to provide another form of orthotic device.

### DETAILED DESCRIPTION OF THE INVENTION

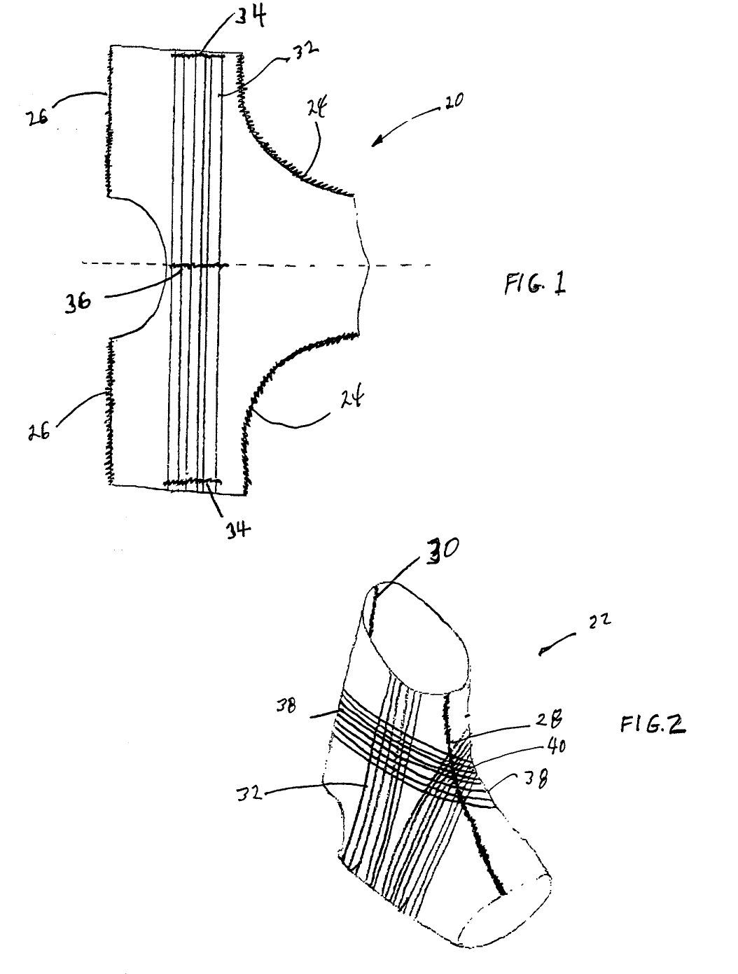

Referring to FIG. 1, it will be seen that for formation of the orthotic device presented herein, there is provided a panel 20 from which is formed an inner sleeve 22 (FIG. 2). The panel 20 is configured to provide inner sleeve instep hems 24 and Achilles hems 26. The hems 24 are joined together, as by sewing, to interconnect the hems 24 along an inseam line 28. Similarly, the hems 26 are joined together to interconnect the hems 26 along an Achilles line 30. Alternatively, the hems 24, 26 may be provided with strips of hermaphroditic fastener material for quick and easy joining.

The panel 20 is provided with reinforcing strands 32 of material which renders the strands readily bendable but inelastic axially. This property enables the strands 32 to protect against excessive flexing of a joint, by bending to conform to anatomy but not extending axially, that is, refraining from extending in directions in which the device should not yield. The strands 32 may be applied to the panel 20 before the hems 24, 26 are joined together to form the sleeve 22. The strands 32 are tacked to the panel 20, as by stapling or sewing at selected points, such as at ends thereof along end tack lines 34, and preferably a central tack line 36. A figure-eight strand 38 may be applied to the sleeve 22 (FIG. 2) and may be tacked to the sleeve in a central area 40 of the figure-eight.

The panel 20, and therefore the sleeve 22, is of an elastomeric material. Materials found appropriate include latex, nitril, vinyl, and apparel fabric including an elastane fiber. The reinforcing strands are of a material which provides the aforesaid attributes of being bendable but axially inelastic. Materials found appropriate include glass fibers, carbon fibers, and Kevlar® fibers.

The sleeve 22 is then fitted onto the appropriate body part of the patient. In the illustrations, the body part for which the orthotic device is formed is a human foot and ankle.

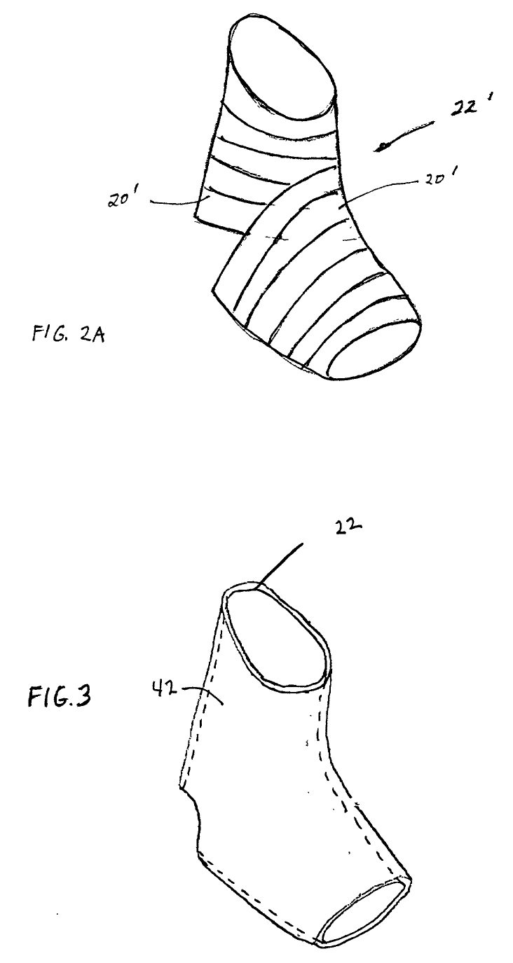

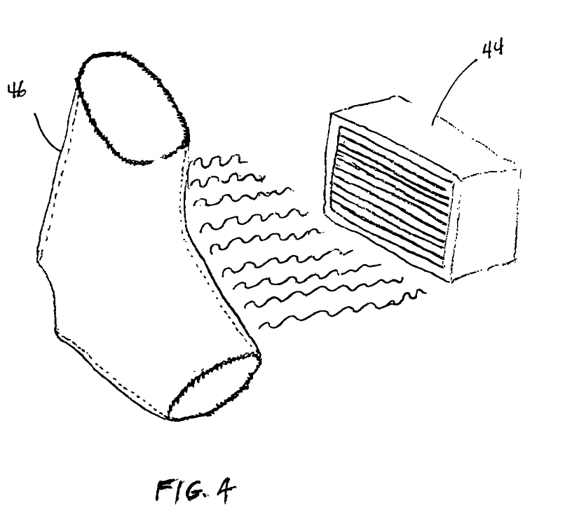

The sleeve 22 and strands 32, 38 are then coated, as by spraying or painting, with a layer of resinous curable material to provide an outer coating 42 (FIG. 3).

The device as shown in FIG. 3 is then subjected to a curing operation, such as the application of heat, to cure the covering 42. The covering 42 may be cured chemically, or by ultraviolet light, or by a common heater or ultra violet light source 44, the latter two alternatives being shown diagrammatically in FIG. 4, as an example.

As the covering 42 cures, it forms a rigid device closely conforming to the shape to which it is applied and is supportive of that body area. The tacking of the strands 32 to the sleeve 22 serve to keep the strands in place during application of the resinous material. The strands are attached to the resin along their entire lengths, providing supporting strength to the resinous material.

The resinous material of the outer covering 42 may be selected from a number of materials found to perform appropriately. The materials known to be acceptable include curable epoxy, acrylates (such as urethane acrylate, 2-phenoxyethyl acrylate, di-trimethylopropane tetraacrylate, dipentaerythritol pentaacrylate), urethane (including two component urethane and two component urethane elastromers), silicone, copolymers thereof and polymer alloys thereof, and thermoplastics with glass transition temperatures in a range of 120-160° F. In a preferred embodiment, the resinous material comprises a polymer exhibiting a cure time of about five minutes or less.

If desired, the panel 20 and reinforcing strands 32 may be provided in an integrated fashion. For example, if the panel 20 is of a fabric material, the strands 32 may be woven into the fabric; if the panel 20 is of latex, or the like, the strands may be embedded therein. The integrated panel and strands appear much the same as the panel and strands tacked thereon, as shown in FIGS. 1 and 2.

In an alternative embodiment, and particularly for use to prevent injury, the strands may be omitted. In this embodiment, the covering 42 is deposited on the sleeve 22, as shown in FIG. 3, but without reinforcing strands integrated with, or attached to, the sleeve 22. Again, curing of the covering 42 serves to harden the device and conform the device to the substrate body part.

In another alternative embodiment, the sleeve 22 may be formed by the sleeve material in tape form 20′ (FIG. 2A) and then coated with the covering 42, as shown in FIG. 3.

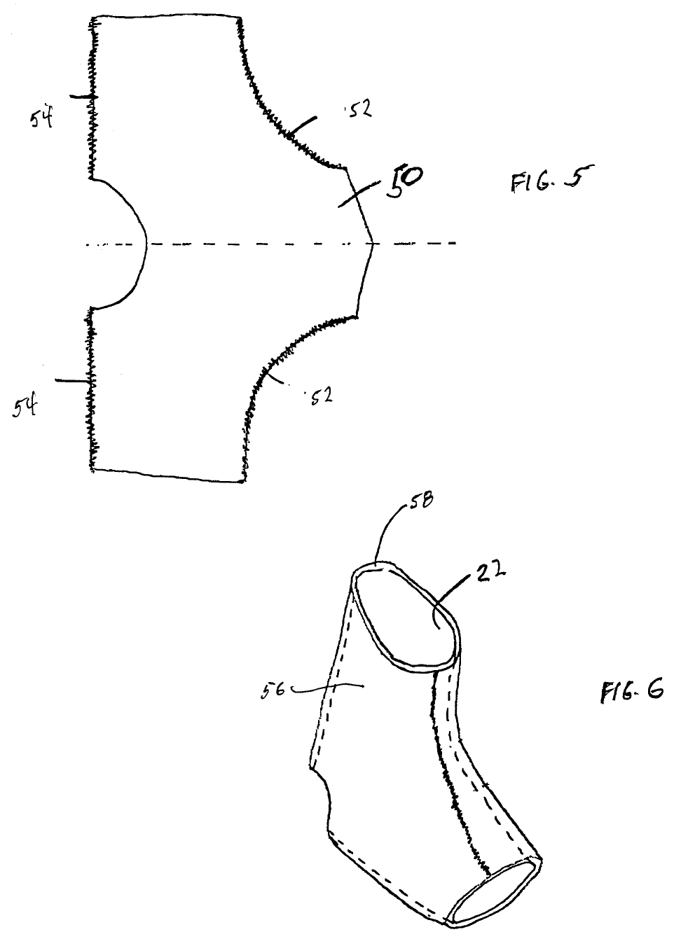

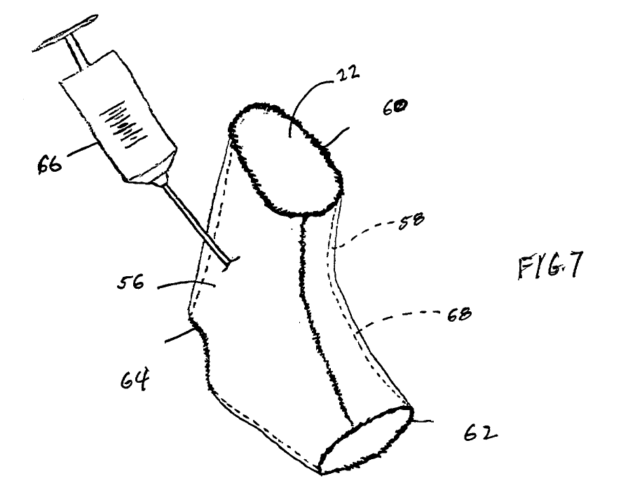

In a further alternative embodiment, there is provided a second panel 50 (FIG. 5) provided with hems 52, 54, similar to the hems 24, 26. The second panel 50 is of a size slightly larger than that of the panel 20, such that the second panel 50 can be fitted and joined together, similarly to the first panel 50, over the device shown in FIG. 2. That is, the panel 50 is fitted over the inner sleeve 22 and strands 32, 38 to form an outer sleeve 56 over the inner sleeve 22. The two sleeves 22, 56 define a space 58 therebetween.

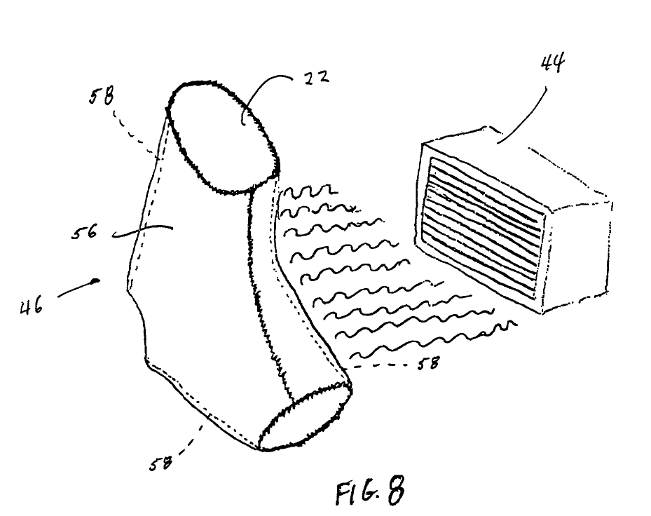

As shown in FIG. 7, the space 58 is closed at ends 60, 62, 64 and the space 58 is filled, as by injection by a syringe 66, with the aforementioned resinous material, which in this embodiment constitutes a middle layer 68 of a laminar structure, rather than the outer covering 42 heretofore described.

If a two part material is used for the middle layer 68, such as two-component urethanes or two component urethane elastomers, the injection device may comprise a self-mixing liquid polymer injector. In such instances, the outer sleeve 56 may be provided with one or more one-way valves (not shown).

The assembly as shown in FIG. 7 is then subjected to heat or ultra violet light (FIG. 8) in the same manner as the previously described device 46, illustrated in FIG. 4. Upon curing of the resinous material, the middle layer 68 hardens and the inner and outer elastomeric sleeves 22, 58, permit the device to assume a configuration conforming to the foot and ankle upon which the device is mounted. The finished device is about ⅛ inch in thickness and therefore not a hindrance when used to protect against injury.

There is thus provided an orthotic device which can be closely conformed to a body area of a patient and which provides required support to either prevent injury or protect a previously injured area. Further, the device can be applied by the patient and cured in little time without the assistance of others.

It will be understood that many additional changes in the details, materials, steps and arrangement of parts, which have been herein described and illustrated in order to explain the nature of the invention, may be made by those skilled in the art within the principles and scope of the invention as expressed in the appended claims.

What is claimed is:

1. An orthotic device for application to a limb of a mammal, the device comprising:
a sleeve for disposition on the limb;
reinforcing strands fixed to said sleeve; and
a curable layer of resin disposed on said sleeve and said strands;
wherein upon curing of said resin said device is conformed to the limb and contains said strands therein.

2. The device in accordance with claim 1 wherein said sleeve is of an elastomeric material.

3. The device in accordance with claim 2 wherein the elastomeric material comprises a material selected from latex, nitrile, vinyl, and apparel fabric including an elastane fiber.

4. The device in accordance with claim 3 wherein said reinforcing strands comprise a selected one of glass fibers, carbon fibers, and Kevlar® fibers.

5. The device in accordance with claim 4 wherein said resin comprises a selected one of epoxy, acrylate, urethane, and silicone materials, copolymers thereof, polymer alloys thereof, and thermoplastics with glass transition temperature in a range of 120-160° F.

6. The device in accordance with claim 1 wherein said resin comprises a selected one of epoxy, acrylate, urethane, and silicone materials, copolymers thereof, polymer alloys thereof, and thermoplastics with glass transition temperature in a range of 120-160° F.

7. The device in accordance with claim 1 wherein said selected resin is curable by a selected one of ultraviolet light, heat, and chemical reaction.

8. The device in accordance with claim 1 wherein said resin comprises a polymer exhibiting a cure time of no more than about five minutes.

9. The device in accordance with claim 4 wherein said reinforcing strands are bendable but are inelastic axially.

10. The device in accordance with claim 9 wherein said reinforcing strands are tacked to said sleeve at selected points and attached to said resin along their entire lengths.

11. The device in accordance with claim 10 wherein said strands are sewn to said sleeve.

12. An orthotic device for application to a limb of a mammal, the device comprising:
an inner sleeve for disposition on the limb;
reinforcing strands fixed to said inner sleeve;
an outer sleeve fixed to and disposed around said inner sleeve, said inner and outer sleeves defining a space therebetween; and
a curable resin disposed in the space;
wherein upon curing of said resin said device is conformed to the limb.

13. The device in accordance with claim 12 wherein said sleeves are each of an elastomeric material.

14. The device in accordance with claim 13 wherein the elastomeric material comprises a material selected from latex, nitrile, vinyl, and apparel fabric including an elastane fiber.

15. The device in accordance with claim 14 wherein said reinforcing strands comprise a selected one of glass fibers, carbon fibers, and Kevlar® fibers.

16. The device in accordance with claim 15 wherein said resin comprises a selected one of epoxy, acrylate, urethane, and silicone materials, copolymers thereof, polymer alloys thereof, and thermoplastics with glass transition temperatures in a range of 120-160° F.

17. The device in accordance with claim 12 wherein said resin comprises a selected one of epoxy, acrylate, urethane, and silicone materials, copolymers thereof, polymer alloys thereof, and thermoplastics with glass transition temperatures in a range of 120-160° F.

18. The device in accordance with claim 12 wherein said selected resin is curable by a selected one of ultraviolet light, heat and chemical reaction.

19. The device in accordance with claim 12 wherein said resin comprises a polymer exhibiting a cure time of no more than about five minutes.

20. The device in accordance with claim 15 wherein said reinforcing strands are bendable but are inelastic axially.

21. The device in accordance with claim 20 wherein said reinforcing strands are tacked to said sleeve at selected points and attached to said resin along their entire lengths.

22. A method for forming a custom orthotic device, the method comprising the steps of:
providing a sleeve of elastomeric material;
tacking reinforcing strands to the sleeve;
positioning the sleeve around a limb of a mammal;
applying a layer of curable resin on the sleeve and over the reinforcing strands;
curing the resin;
wherein the resin is made to harden conforming to the limb.

23. The method in accordance with claim 22 wherein the elastomeric material comprises a material selected from latex, nitrile, vinyl, and apparel fabric including an elastane fiber.

24. The method in accordance with claim 22 wherein the resin comprises a selected one of epoxy, acrylate, urethane, and silicone materials, copolymers thereof, polymer alloys thereof, and thermoplastics with glass transition temperature in a range of 120-160° F.

25. The method in accordance with claim 22 wherein said selected resin is cured by a selected one of ultraviolet light, heat and chemical reaction.

26. The method in accordance with claim 22 wherein said resin comprises a polymer exhibiting a cure time of no more than about five minutes.

27. The method in accordance with claim 22 wherein said reinforcing strands are bendable but are inelastic axially.

28. A method for forming a custom orthotic device, the method comprising the steps of:
providing an inner sleeve of elastomeric material;
tacking reinforcing strands to the sleeve;
fixing an outer sleeve of elastomeric material onto and around the inner sleeve such that the inner and outer sleeves define a space therebetween;
positioning the device thus formed around a limb of a mammal;
injecting curable liquid resin into the space; and
curing the resin;
wherein to harden the resin conforming to the limb.

29. The method in accordance with claim 28 wherein the elastomeric material comprises a material selected from latex, nitrile, vinyl, and apparel fabric including an elastane fiber.

30. The method in accordance with claim 28 wherein the resin comprises a selected one of epoxy, acrylate, urethane, and silicone materials, copolymers thereof, polymer alloys thereof, and thermoplastics with glass transition temperatures in a range of 120-160° F.

31. The method in accordance with claim 28 wherein the selected resin is cured by a selected one of ultraviolet light, heat and chemical reaction.

32. The method in accordance with claim 28 wherein said resin comprises a polymer exhibiting a cure time of no more than about five minutes.

33. The method in accordance with claim 28 wherein the reinforcing strands are bendable but are inelastic axially.

34. The method in accordance with claim 33 wherein the reinforcing strands are tacked to the sleeve at selected points and are attached to the resin along entire lengths of the strands.

35. An orthotic device for application to a limb of a mammal, the device comprising:
a sleeve for disposition on the limb; and
a curable layer of resin disposed on said sleeve;
wherein upon curing of said resin said device is conformed to the limb.

36. The device in accordance with claim 35 wherein said sleeve comprises material in tape form disposed around the limb.

37. An orthotic device for application to a limb of a mammal, the device comprising:
a sleeve for disposition on the limb, said sleeve comprising a sheet of flexible material and reinforcing strands fixed to said sleeve; and
a curable layer of resin disposed on said sleeve;
wherein upon curing of said resin said device is conformed to the limb and contains said strands therein.

38. The device in accordance with claim 37 wherein said sleeve is of an elastomeric material and said strands are embedded therein.

39. The device in accordance with claim 37 wherein said sleeve is of woven fabric and said strands are woven into the fabric.
